(19) United States
(12) Patent Application Publication
(10) Pub. No.: US 2024/0023860 A1
(43) Pub. Date: Jan. 25, 2024

(54) URINE AMOUNT MEASURING DEVICE

(71) Applicants: Akira HORIGUCHI, Takarazuka-shi (JP); Kazutaka YOSHINO, Kobe-shi (JP)

(72) Inventors: Akira HORIGUCHI, Takarazuka-shi (JP); Kazutaka YOSHINO, Kobe-shi (JP)

(21) Appl. No.: 17/757,917

(22) PCT Filed: Mar. 4, 2021
(86) PCT No.: PCT/JP2021/008495

§ 371 Date: Sep. 9, 2022

(51) Int. Cl. A61B 5/20; A61B 5/00

(52) U.S. Cl. CPC A61B 5/208; A61B 5/6891

(57) ABSTRACT

The urine amount measuring device (100) includes a container-side support portion (10), a sensor (32), a housing (101), a first fixing portion (20), and a second fixing portion (60). The container-side support portion (10) supports the suspension portion (50) on which a urine storage container (C) is hung. The urine storage container (C) stores urine of a person lying on the bed (B). The sensor (32) outputs a signal that changes according to change in force acting on the container-side support portion (10). The housing (101) houses the sensor (32). The first fixing portion (20) fixes a tubular member (T) to the housing (101). The tubular member (T) allows the urine to flow from the person to the urine storage container (C). The second fixing portion (60) fixes the tubular member (T) to the suspension portion (50).

### TECHNICAL FIELD

The present invention relates to a urine amount measuring device.

### BACKGROUND ART

A known urine amount measuring device measures the amount of urine collected in a urine storage container (see Patent Document 1, for example). Patent Document 1 discloses a digital waste liquid measuring machine (urine amount measuring device). The digital waste liquid measuring machine of Patent Document 1 includes a base unit that capable of being fixed to a bed and a cantilever load cell. The base unit is fixed in place on the bed so that the cantilever load cell is approximately horizontal. A hook is provided at a measurement end of the cantilever load cell. A fixed end of the cantilever load cell is cantilevered from the base unit. A waste liquid bag (urine storage container) communicating with a drainage tube (tube-shaped member) is hung on the hook.

### CITATION LIST

### Patent Literature

Patent Literature 1

Japanese Unexamined Patent Publication No. 5-305147

### SUMMARY OF INVENTION

### Technical Problem

However, in the digital waste liquid measuring machine (urine amount measuring device) of Patent Document 1, movement of the drainage tube (tube-shaped member) caused by the movement of a patient may result in swing of the waste liquid bag (urine storage container). Movement of the drainage tube caused by being touched by something may also result in swing of the waste liquid bag. For example, movement of the drainage tube caused by being touched by a nurse may result in swing of the waste liquid bag. Once the waste liquid bag swings, the force acts on the measurement end of the load cell, which may reduce the measurement accuracy of the urine amount.

The present invention has been made in view of the above problems, and an object of the present invention is to provide a urine amount measuring device in which decrease in the measurement accuracy of the urine amount hardly occurs.

### Solution to Problem

In the present invention, the urine amount measuring device includes a container-side support portion, a sensor, a housing, a first fixing portion, and a second fixing portion. The container-side support portion supports a suspension portion for hanging the urine storage container. The urine storage container stores urine of a person lying on a bed. The sensor outputs a signal that changes according to the change in force acting on the container-side support portion. The housing houses the sensor. The first fixing portion fixes the tubular member to the housing. The tubular member allows the urine to flow from the person to the urine storage container. The second fixing portion fixes the tubular member to the suspension portion.

In some embodiments, the container-side support portion supports the suspension portion in a freely swinging manner.

In some embodiments, the urine amount measuring device further comprises the suspension portion.

In some embodiments, the suspension portion supports the urine storage container in a freely swinging manner.

In some embodiments, the first fixing portion is freely detachably connected to the housing.

In some embodiments, the housing includes connecting portions at a plurality of positions where the first fixing portion is to be connected.

In some embodiments, the housing includes a mounting portion to be mounted on a bed-side support portion. The bed-side support portion is to be supported by the bed.

In some embodiments, the urine amount measuring device further includes the bed-side support portion.

In some embodiments, the bed-side support portion includes a belt member and a belt support member. The belt member is to be attached to the mounting portion. The belt support member is to be supported by the bed. The belt member is hung from the belt support member.

In some embodiments, the bed-side support includes a belt member to be supported by the bed. The belt member is to be attached to the mounting portion.

In some embodiments, the mounting portion is provided on a rear surface of the housing.

In some embodiments, the mounting portion is provided on a top surface of the housing.

In some embodiments, the bed-side support portion includes a housing support portion for supporting the housing and a base portion for connecting the housing support portion. The base portion is to be supported by the bed. The housing support portion is to be attached to the mounting portion.

In some embodiments, the urine amount measuring device further includes a gripping member to grip the bed.

### Advantageous Effects of Invention

According to the urine amount measuring device of the present invention, decrease in the measurement accuracy of the urine amount hardly occurs.

### BRIEF DESCRIPTION OF DRAWINGS

FIG. 1A is a perspective view of a urine amount measuring device according to a first embodiment of the present invention.

FIG. 1B is a perspective view of the urine amount measuring device as viewed from a direction different from that of FIG. 1A.

FIG. 2A is a perspective view of the urine amount measuring device as viewed from a direction different from those of FIGS. 1A and 1B.

FIG. 2B is a perspective view of the urine amount measuring device as viewed from a direction different from those of FIGS. 1A, 1B and 2A.

FIG. 3A is a perspective view of the urine amount measuring device as viewed from the rear side thereof.

FIG. 3B is a perspective view of the urine amount measuring device as viewed from the front side thereof.

FIG. 4 is a side view of the urine amount measuring device according to the embodiment of the present invention.

FIG. 5 illustrates the urine amount measuring device attached to a bed.

FIG. 6 is a block diagram of a configuration of the urine amount measuring device according to the embodiment of the present invention.

FIG. 7 illustrates a first application of the urine amount measuring device according to the embodiment of the present invention.

FIG. 8 is a front view of a part of the urine amount measuring device according to the embodiment of the present invention.

FIG. 9 illustrates a second application of the urine amount measuring device according to the embodiment of the present invention.

FIG. 10A illustrates a part of the bed.

FIG. 10B illustrates a belt support member included in the urine amount measuring device according to the embodiment of the present invention.

FIG. 11A illustrates the bed and a housing support frame.

FIG. 11B is an enlarged view illustrating the bed and the housing support frame.

FIG. 12A is a perspective view of the housing support frame.

FIG. 12B is a perspective view of the urine amount measuring device as viewed from the rear side thereof.

FIG. 13A is a perspective view of the urine amount measuring device as viewed from the rear side thereof.

FIG. 13B is a perspective view of the urine amount measuring device as viewed from the rear side thereof.

FIG. 14 illustrates a variation of the urine amount measuring device according to the embodiment of the present invention.

FIG. 15A is a side view of a urine amount measuring device according to a second embodiment of the present invention.

FIG. 15B illustrates a part of the bed.

### DESCRIPTION OF EMBODIMENTS

The following describes embodiments of a urine amount measuring device according to the present invention with reference to the drawings. However, the present invention is not limited to the following embodiments. Note that duplicated descriptions may be omitted as appropriate. In the drawings, the same or corresponding parts are denoted by the same reference signs, and description thereof will not be repeated.

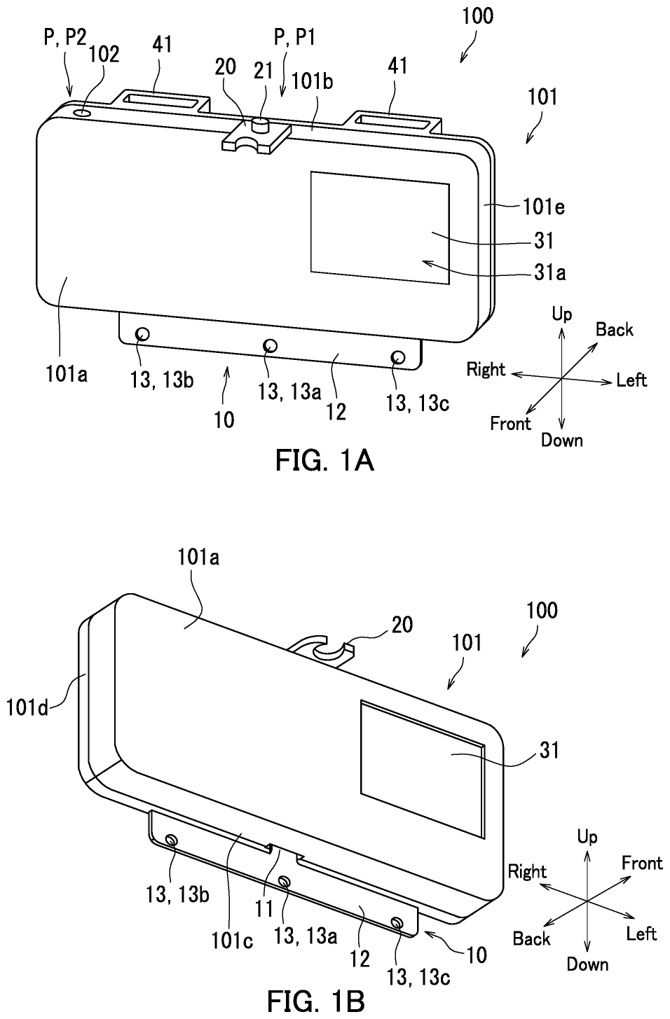

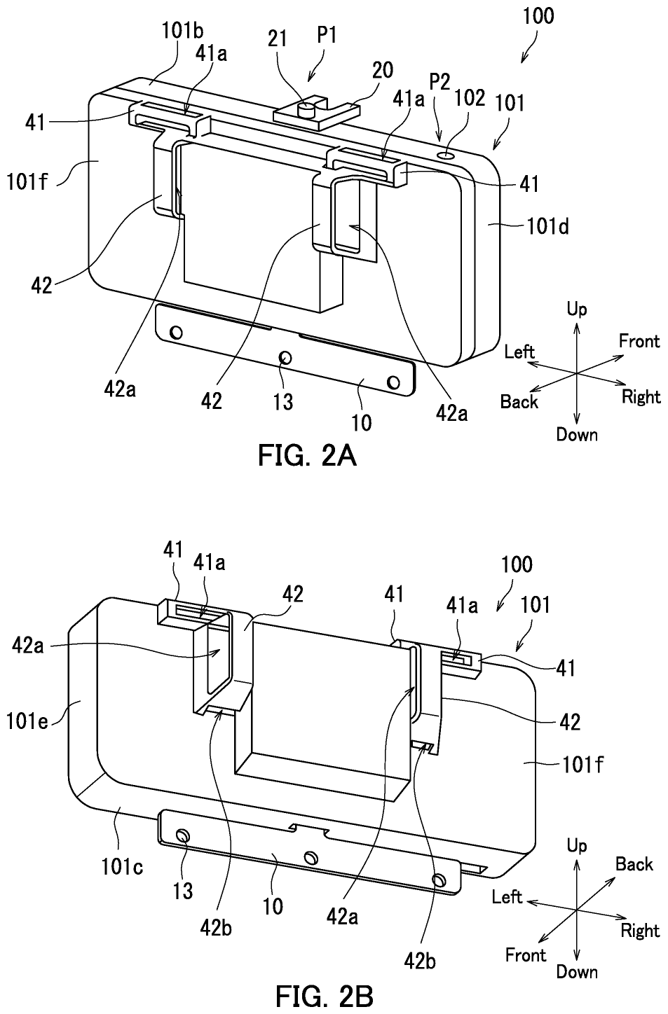

A urine amount measuring device 100 of a first embodiment will now be described with reference to FIGS. 1A, 1B, 2A, and 2B. FIG. 1A is a perspective view of the urine amount measuring device 100 of the present embodiment. FIG. 1B is a perspective view of the urine amount measuring device 100 as viewed from a direction different from that of FIG. 1A. FIG. 2A is a perspective view of the urine amount measuring device 100 as viewed from a direction different from those of FIGS. 1A and 1B. FIG. 2B is a perspective view of the urine amount measuring device 100 as viewed from a direction different from those of FIGS. 1A, 1B and 2A. Specifically, FIGS. 1A and 1B illustrate the urine amount measuring device 100 as viewed from the front side thereof. FIGS. 2A and 2B illustrate the urine amount measuring device 100 as viewed from the rear side thereof.

The urine amount measuring device 100 measures the amount of urine. As illustrated in FIGS. 1A and 1B, the urine amount measuring device 100 includes a container-side support portion 10, a first fixing portion 20, a fastening portion 21, a display section 31, and a housing 101. In the present embodiment, the side on which the display section 31 is disposed is referred to as “front side of the urine amount measuring device 100”, and the opposite side of the front side is referred to as “rear side of the urine amount measuring device 100”. The right side as viewed from the front side is referred to as “left side of the urine amount measuring device 100”, and the left side as viewed from the front side is referred to as “right side of the urine amount measuring device 100”. The side on which the container-side support portion 10 is disposed is referred to as “lower side of the urine amount measuring device 100”, and the opposite side of the lower side is referred to as “upper side of the urine amount measuring device 100”.

The housing 101 forms a space inside. The housing 101 is, for example, box-shaped. As illustrated in FIG. 1A, the housing 101 has a front surface 101a, a top surface 101b, and a left side surface 101e (one of side surfaces). As illustrated in FIG. 1B, the housing 101 further has a right side surface 101d (another side surface) and a bottom surface 101c. In the present embodiment, the housing 101 has a substantially rectangular parallelepiped shape. Note that the housing 101 includes two first mounting portions 41 as illustrated in FIG. 1A. The first mounting portions 41 will be described later with reference to FIG. 2A.

As illustrated in FIG. 1A, the display section 31 includes a display surface 31a. The display surface 31a is exposed from the front surface 101a of the housing 101. The display section 31 displays a measurement result of the urine amount on the display surface 31a. The display section 31 is, for example, a liquid crystal display or an organic EL display. For example, when the display section 31 is a liquid crystal display, the liquid crystal display is disposed inside the housing 101 except the display surface of the liquid crystal panel. For example, the liquid crystal display may be supported by the housing 101 from the inside of the housing 101.

The fastening portion 21 connects the first fixing portion 20 to the housing 101. Specifically, the first fixing portion 20 is connected to the housing 101 by fastening the fastening portion 21 to the housing 101. More specifically, the fastening portion 21 penetrates the first fixing portion 20 to be fastened to the housing 101. The first fixing portion 20 has a through hole for insertion of the fastening portion 21. The fastening portion 21 is a bolt or a screw, for example.

The first fixing portion 20 is connected to the top surface 101b of the housing 101. In the present embodiment, the first fixing portion 20 is freely detachably connected to the housing 101. Specifically, the first fixing portion 20 can be removed from the housing 101 by releasing fastening of the fastening portion 21 from the housing 101.

The housing 101 includes connecting portions P on the top surface 101b at a plurality of positions where the first fixing portion 20 is to be connected. In the present embodiment, the connecting portions P include a first connecting portion P1 and a second connecting portion P2. The first connecting portion P1 and the second connecting portion P2 each have a fastening hole 102 formed in the top wall of the housing 101. The fastening portion 21 is fastened in the fastening hole 102.

The first connecting portion P1 is located at the center of the housing 101 in the left-right direction. The second connecting portion P2 is located near the right side surface 101d of the housing 101. In other words, the second connecting portion P2 is located at the right end portion of the top surface 101b of the housing 101.

As illustrated in FIG. 1B, the container-side support portion 10 protrudes downward of the housing 101 from the bottom surface 101c of the housing. In the present embodiment, the container-side support portion 10 has a plate shape.

The container-side support portion 10 includes a coupling portion 11. The coupling portion 11 protrudes downward of the housing 101 from the bottom surface 101c of the housing 101. In detail, the coupling portion 11 is inserted into a through hole formed in the bottom wall of the housing 101 and protrudes from the inside to the outside of the housing 101. The upper end of the coupling portion 11 is located inside the housing 101, and the lower side of the coupling portion 11 is located below the housing 101.

The container-side support portion 10 further includes a holding portion 12. The holding portion 12 is disposed below the bottom surface 101c of the housing 101 and is combined with the lower end of the coupling portion 11. In the present embodiment, the holding portion 12 extends in the left and right directions from a point where the holding portion 12 is combined with the coupling portion 11.

As illustrated in FIGS. 1A and 1B, the holding portion 12 has a plurality of hooking holes 13. Each of the hooking holes 13 penetrates the holding portion 12. The hooking holes 13 are arranged in the left-right direction (extension direction of the holding portion 12). In the present embodiment, the hooking holes 13 include a first hooking hole 13a, a second hooking hole 13b, and a third hooking hole 13c.

The first hooking hole 13a is located at the center of the holding portion 12 in the left-right direction. The second hooking hole 13b is located on the right side of the first hooking hole 13a. The third hooking hole 13c is located on the left side of the first hooking hole 13a. The distance between the second hooking hole 13b and the first hooking hole 13a is approximately equal to the distance between the third hooking hole 13c and the first hooking hole 13a. In other words, the second hooking hole 13b and the third hooking hole 13c are respectively arranged at right and left positions each separated from the center of the holding portion 12 in the left and right directions by approximately equal distances.

The following describes the urine amount measuring device 100 of the present embodiment with reference to FIGS. 2A and 2B. As illustrated in FIGS. 2A and 2B, the housing 101 further has a rear surface 101f. The housing 101 further includes two second mounting portions 42.

As illustrated in FIG. 2A, the two first mounting portions 41 are provided on the rear surface 101f of the housing 101. More specifically, the two first mounting portions 41 are provided on the top of the rear surface 101f. Each of the first mounting portions 41 protrudes rearward from the rear surface 101f. Each of the two first mounting portion 41 has a first insertion hole 41a. The first insertion holes 41a penetrate the first mounting portions 41 from the top surfaces to the rear surfaces of the first mounting portions 41. In other words, the first insertion holes 41a each have openings on the top surface and the rear surface of each first mounting portions 41.

The two first mounting portions 41 are arranged in the left-right direction. One of the two first mounting portions 41 is arranged on the right side of the center of the housing 101 in the left-right direction, while the other is arranged on the left side thereof. The two first mounting portions 41 are arranged at left and right positions each separated from the center of the housing 101 in the left-right direction by approximately equal distances.

As illustrated in FIGS. 2A and 2B, the two second mounting portions 42 are provided on the rear surface 101f of the housing 101. In this embodiment, the two second mounting portions 42 are provided below the respective two first mounting portions 41. Each of the two second mounting portions 42 protrudes rearward from the rear surface 101f. The second mounting portions 42 each have a second insertion hole 42a. The second insertion holes 42a each penetrate the second mounting portions 42 in the left-right direction.

The two second mounting portions 42 are arranged in the left-right direction. One of the second mounting portions 42 is arranged on the right side of the center of the housing 101 in the left-right direction, while the other is arranged on the left side thereof. The two second mounting portions 42 are arranged at left and right positions each separated from the center of the housing 101 in the left-right direction by approximately equal distances.

As illustrated in FIG. 2B, each of the second mounting portions 42 further has an insertion hole 42b. The insertion holes 42b extend in the vertical direction. The insertion holes 42b have openings in the bottom surfaces of the respective second mounting portions 42. The openings in the bottom surfaces of the second mounting portions 42 are located substantially at the center of the housing 101 in the vertical direction.

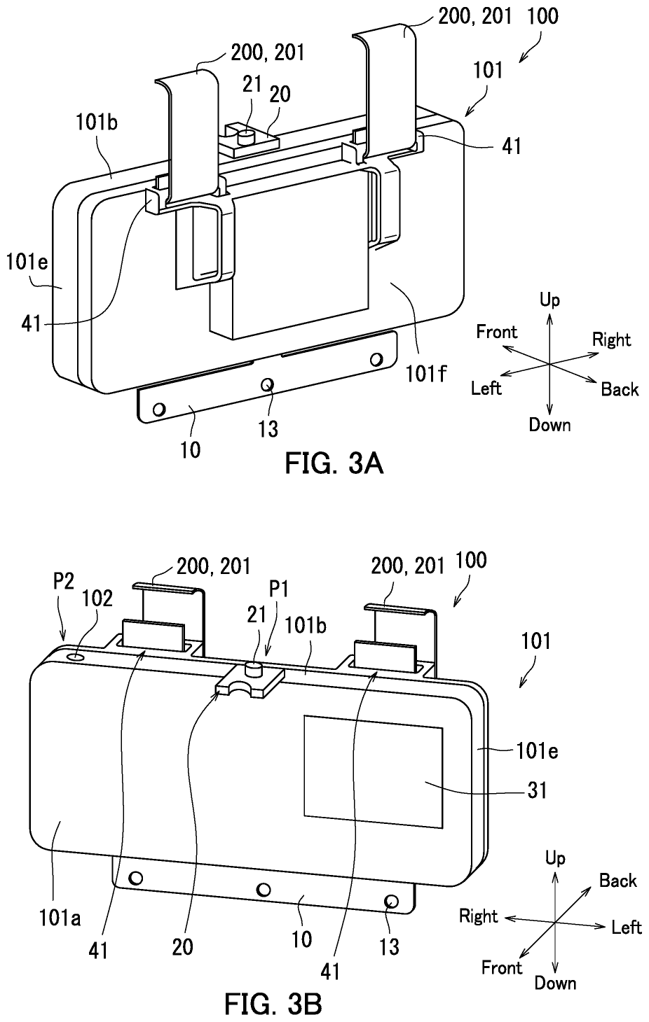

The urine amount measuring device 100 of the present embodiment will now be described with reference to FIGS. 3A and 3B. FIG. 3A is a perspective view of the urine amount measuring device 100 as viewed from the rear side thereof. FIG. 3B is a perspective view of the urine amount measuring device 100 as viewed from the front side thereof.

As illustrated in FIGS. 3A and 3B, the urine amount measuring device 100 further includes a bed-side support portion 200. The bed-side support portion 200 is to be attached to the housing 101. Specifically, the bed-side support portion 200 includes two first belt members 201. The two first belt members 201 are attached to the respective first mounting portions 41. In detail, the first belt members 201 are inserted into the respective first insertion holes 41a described with reference to FIG. 2A. The first belt members 201 each are, for example, a hook-and-loop fastener, binding tape, or a piece of string. Alternatively, the first belt members 201 may include at least two of the hook-and-loop fastener, the binding tape, and the string.

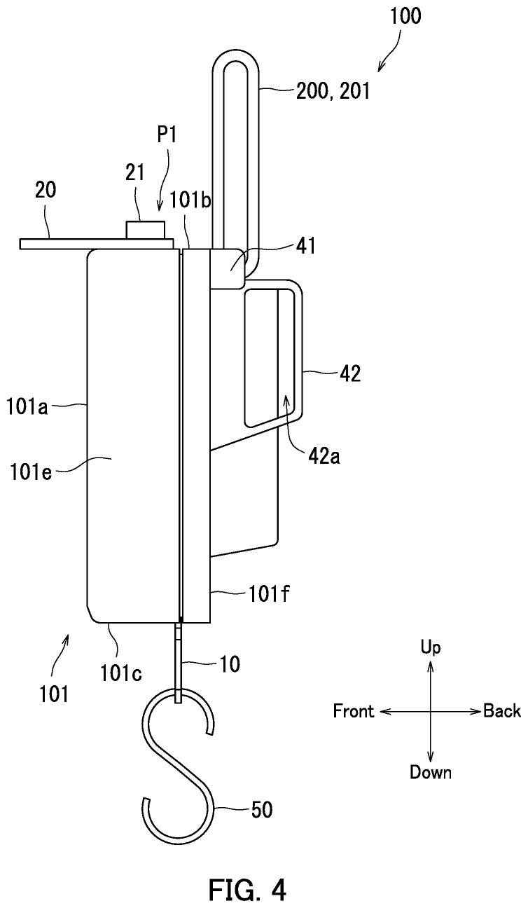

The urine amount measuring device 100 of the present embodiment will now be described with reference to FIG. 4. FIG. 4 is a side view of the urine amount measuring device 100 of the present embodiment. As illustrated in FIG. 4, the urine amount measuring device 100 further includes a suspension portion 50. In the present embodiment, the suspension portion 50 is an S-shaped hook.

The container-side support portion 10 supports the suspension portion 50. In the present embodiment, the container-side support portion 10 supports the suspension portion 50 in a freely swinging manner. In detail, the suspension portion 50 is hooked on one of the hooking holes 13 described with reference to FIGS. 1A and 1B. More specifically, when there is one suspension portion 50, the suspension portion 50 is hooked on the first hooking hole 13a described with reference to FIGS. 1A and 1B.

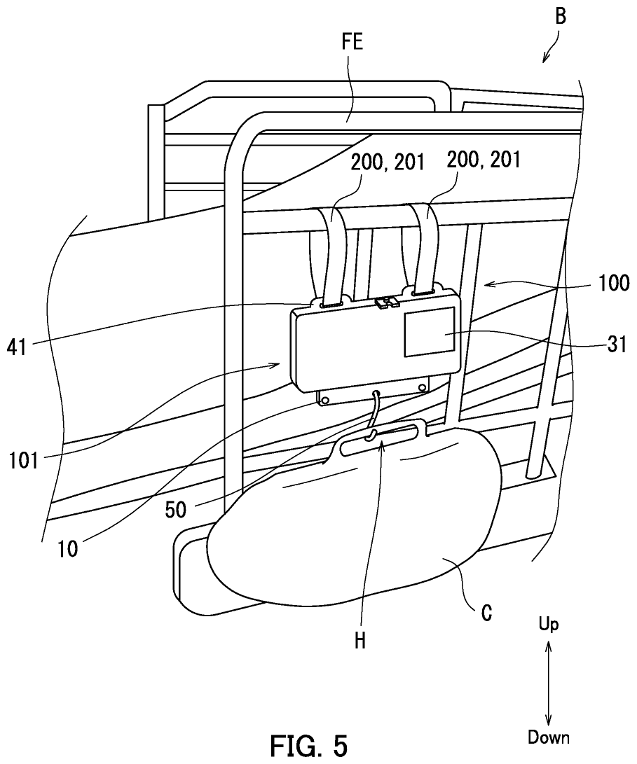

The following describes the urine amount measuring device 100 of the present embodiment with reference to FIG. 5. FIG. 5 illustrates the urine amount measuring device 100 attached to the bed B. In detail, FIG. 5 illustrates a first application of the urine amount measuring device 100. Note that the fastening holes 102 and the first fixing portion 20 are omitted in FIG. 5 for simplification of the drawing.

As illustrated in FIG. 5, the bed-side support portion 200 is supported by the bed B. In the first application, the urine amount measuring device 100 is supported by the bed B in a freely swinging manner. Specifically, the bed B includes a fence FE, and the first belt members 201 are supported by the fence FE of the bed B. As a result, the urine amount measuring device 100 is suspended from the fence FE of the bed B by the first belt members 201.

As illustrated in FIG. 5, a urine storage container C is hung on the suspension portion 50. The urine storage container C stores urine of a person (e.g., a patient) lying on the bed B. In the present embodiment, the suspension portion 50 supports the urine storage container C in a freely swinging manner. Specifically, a holding portion H of the urine storage container C is hooked on the suspension portion 50 (S-shaped hook).

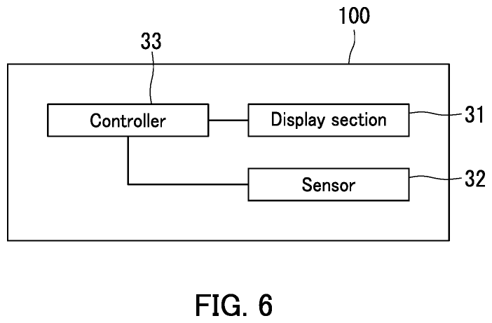

The following describes the urine amount measuring device 100 of the present embodiment with reference to FIGS. 5 and 6. FIG. 6 is a block diagram illustrating the configuration of the urine amount measuring device 100 of the present embodiment. As illustrated in FIG. 6, the urine amount measuring device 100 further includes a sensor 32 and a controller 33. The sensor 32 and the controller 33 are housed in the housing 101 described with reference to FIGS. 1A, 1B, 2A, and 2B.

The sensor 32 outputs a signal that varies according to change in force acting on the container-side support portion 10. The force acting on the container-side support portion 10 changes according to change in the total weight of the urine storage container C hung on the suspension portion 50. The total weight of the urine storage container C is the total value of the weight of the urine storage container C and the weight of the urine stored in the urine storage container C. The total weight of the container-side support portion 10 changes according to the amount of urine stored in the urine storage container C. Therefore, the force acting on the container-side support portion 10 changes according to the amount of urine stored in the urine storage container C.

In detail, the urine storage container C pulls the suspension portion 50 in the gravity direction. The force (tensile force) of the urine storage container C pulling the suspension portion 50 in the gravity direction is transmitted to the container-side support portion 10 via the suspension portion 50, thereby acting on the sensor 32 via the container-side support portion 10.

The force (tensile force) of the urine storage container C pulling the suspension portion 50 in the gravity direction changes according to change in the total weight of the urine storage container C. Therefore, change in amount of urine stored in the urine storage container C result in change inforce acting on the sensor 32 via the container-side support portion 10, thereby causing the signal which is to be output from the sensor 32 to change.

In the present embodiment, the sensor 32 is a cantilever load cell. Specifically, the load cell includes a strain gauge, and the upper end of the coupling portion 11 described with reference to FIG. 1B is connected to the measurement end of the strain gauge.

The controller 33 causes the display section 31 to display the amount of urine stored in the urine storage container C based on the signal output by the sensor 32. The controller 33 may include a microcomputer, for example. Alternatively, the controller 33 may include an analog-to-digital converter (A/D converter), a processor such as a central processing unit (CPU) or a micro processing unit (MPU), and at least one of read-only memory (ROM) and random-access memory (RAM). The A/D converter converts the analog signal output from the sensor 32 to a digital signal.

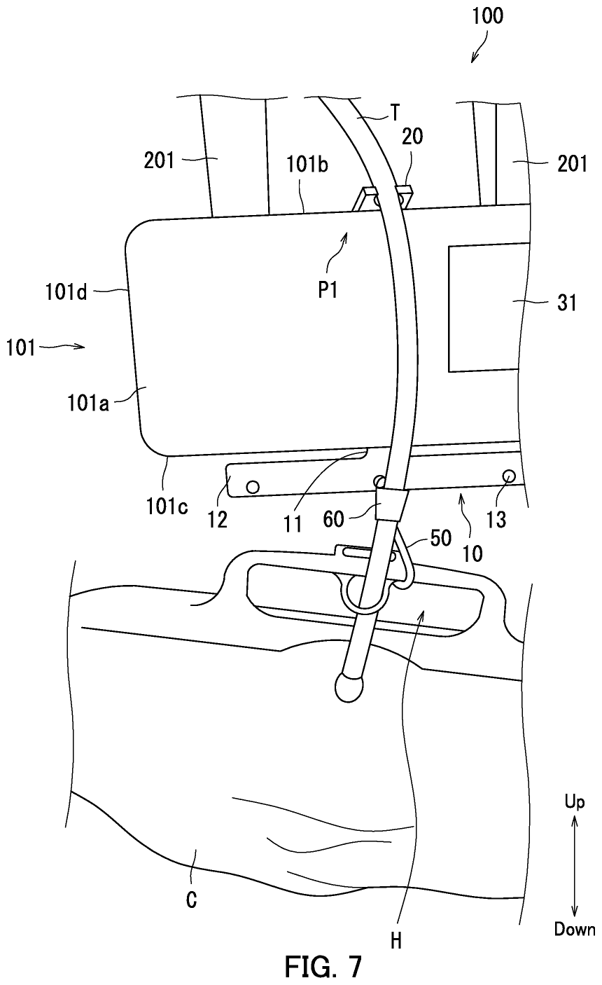

The following describes the urine amount measuring device 100 of the present embodiment with reference to FIG. 7. FIG. 7 illustrates the first application of the urine amount measuring device 100 of the present embodiment. As illustrated in FIG. 7, a first end of the urinary catheter T is connected to the urine storage container C. A second end of the urinary drainage tube T is connected to an indwelling bladder catheter placed in a human bladder, for example. Human urine is stored in the urine storage container C via the urinary catheter tube T. The urinary catheter tube T is an example of a tubular member allows urine to flow from a person to the urine storage container C. The urine amount measuring device 100 measures the amount of urine by measuring the total weight of the urine storage container C.

As illustrated in FIG. 7, the first fixing portion 20 fixes the urinary catheter T to the housing 101. The first fixing portion 20 freely detachably holds the urinary catheter T. In the present embodiment, the first fixing portion 20 grips the urinary catheter tube T.

As illustrated in FIG. 7, in the first application, the first end of the urinary catheter tube T is connected to the central portion of the urine storage container C. Additionally, the first fixing portion 20 is connected to the first connecting portion P1 of the housing 101 in the first application. In other words, the first fixing portion 20 is fixed to the central portion of the housing 101.

As illustrated in FIG. 7, the urine amount measuring device 100 further includes a second fixing portion 60. The second fixing portion 60 fixes the urinary catheter T to the suspension portion 50. The second fixing portion 60 is freely attachable to and freely detachable from the urinary catheter tube T and the suspension portion 50. The second fixing portion 60 is a binding band, a hook-and-loop fastener, or a piece of string, for example. Alternatively, the second fixing portion 60 may include at least two of the binding band, the hook-and-loop fastener, and the string.

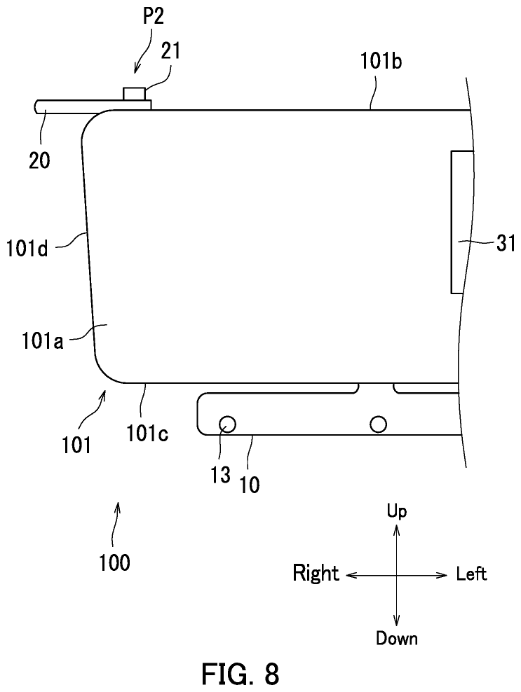

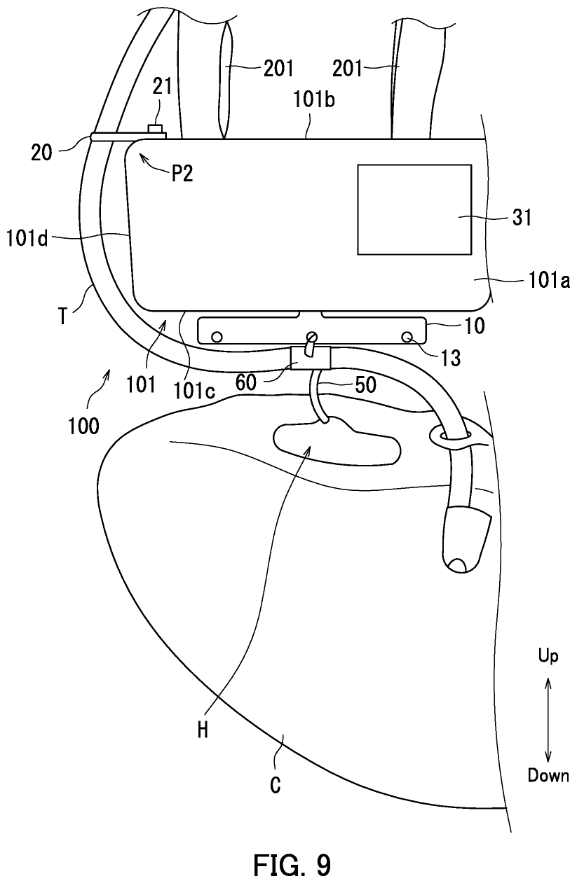

A second application of the urine amount measuring device 100 of the present embodiment will now be described with reference to FIGS. 8 and 9. FIG. 8 is a front view of a part of the urine amount measuring device 100 of the present embodiment.

As described with reference to FIG. 1A, the first fixing portion 20 is freely attachable to and detachable from the housing 101 and is connected to the first connecting portion P1 or the second connecting portion P2 of the housing 101. As illustrated in FIG. 8, in the second application, the first fixing portion 20 is connected to the second connecting portion P2 of the housing 101.

FIG. 9 illustrates the second application of the urine amount measuring device 100 of the present embodiment. As illustrated in FIG. 9, in the second application, the first end of the urinary catheter tube T is connected to the right side portion of the urine storage container C.

As described above with reference to FIGS. 1A to 9, according to the present embodiment, the urinary catheter T is fixed to the housing 101 by the first fixing portion 20. The urinary catheter T is fixed to the suspension portion 50 by the second fixing portion 60. As a result, decrease in accuracy of urine amount measurement hardly occurs.

Specifically, for example, the urine storage container C swings due to movement of the urinary catheter T, which caused by movement of the patient. The swing of the urine storage container C causes the urine storage container C to pull the suspension portion 50, which change the force acting on the container-side support portion 10. Therefore, movement of the patient result in change of the signal to be output from the sensor 32. The accuracy of urine amount measurement decreases because of influenced by such change of the signal. The urinary catheter T moves when touched by something or someone (e.g., a nurse), thereby causing the urine storage container C to swing. In this case, the signal to be output from the sensor 32 also changes. However, according to the present embodiment, since the urinary drainage tube T is fixed by the first fixing portion 20 and the second fixing portion 60, the urine storage container C hardly swings even in a situation in which the patient moves or the urinary drainage tube T is touched by something or somebody (e.g., a nurse). Therefore, decrease in accuracy of urine amount measurement hardly occurs according to the present embodiment.

Additionally, the position where the first fixing portion 20 is fixed to the housing 101 is variable according to the present embodiment. Accordingly, the position where the urinary catheter T is fixed to the housing 101 can be changed according to the type of the urine storage container C to be used. Therefore, the urine amount measuring device 100 of the present embodiment can be used for various types of urine storage containers C.

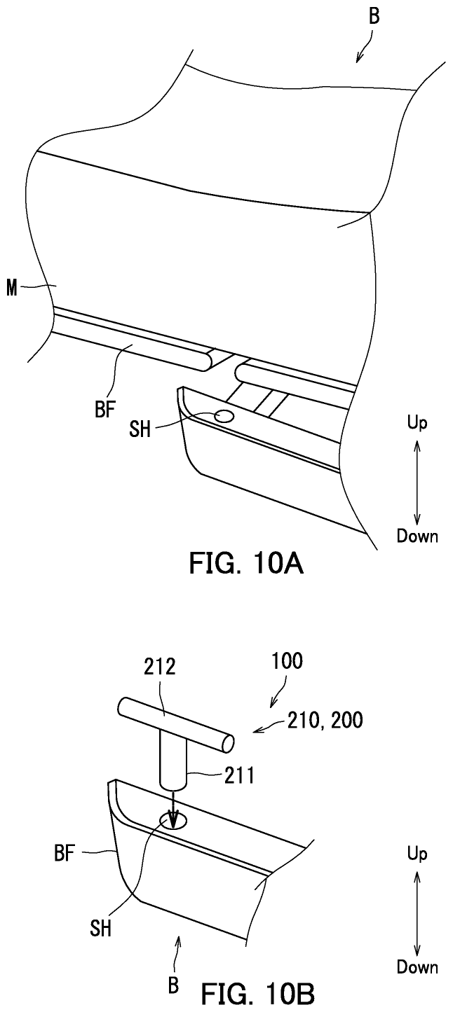

The urine amount measuring device 100 of the present embodiment will be further described with reference to FIGS. 10A and 10B. FIG. 10A illustrates a part of the bed B. In detail, FIG. 10A illustrates a part of bed B in an enlarged scale. FIG. 10B illustrates a belt support member 210 included in the urine amount measuring device 100 of the present embodiment.

As illustrated in FIG. 10A, the bed B incudes a mattress M and a bed frame BF. The bed frame BF supports the mattress M. The bed frame BF has a hole SH extending in the vertical direction, as in the example illustrated in FIG. 10A.

As illustrated in FIG. 10B, the bed-side support portion 200 further includes a belt support member 210. The belt support member 210 is fitted in the hole SH of the bed frame BF. Specifically, the belt support member 210 has a belt support portion 212 and a rod-shaped portion 211 extending in the vertical direction.

The rod-shaped portion 211 is inserted into the hole SH of the bed frame BF. As a result, the belt support member 210 is held by the bed B (bed frame BF). The belt support portion 212 is coupled to the top end of the rod-shaped portion 211. The belt support portion 212 extends in a direction (transverse direction) perpendicular to the vertical direction. In detail, the belt support portion 212 extends toward the left and right sides from a combining position where the belt support portion 212 is combined with the rod-shaped portion 211. The two first belt members 201 described with reference to FIGS. 3A and 3B are hung on the belt support portion 212.

According to the present embodiment, the urine amount measuring device 100 can be supported by the bed B by using the belt support member 210 even when the bed B does not have the fence FE.

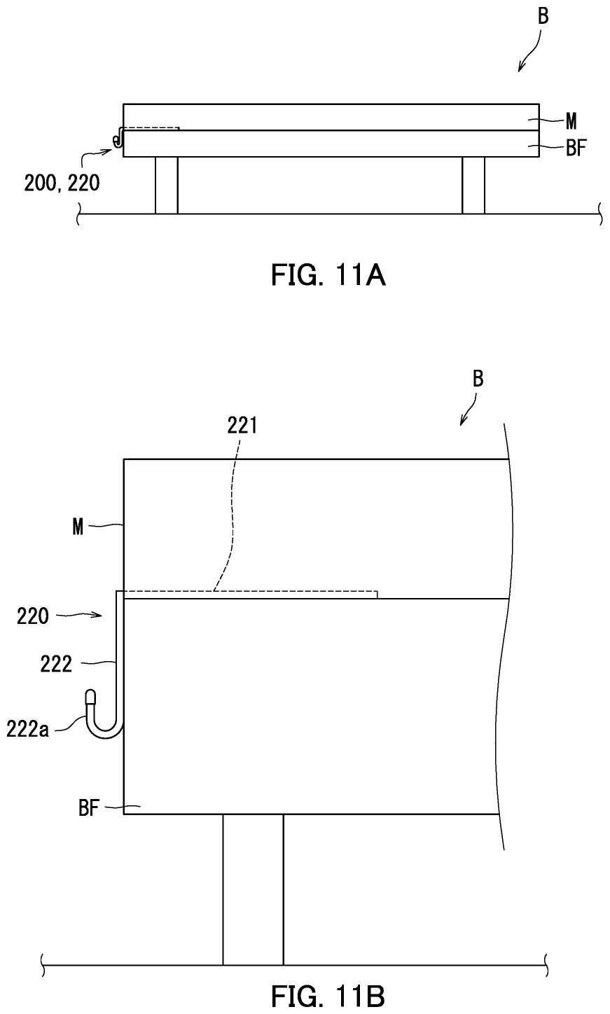

A third application of the urine amount measuring device 100 of the present embodiment will now be described with reference to FIGS. 11A, 11B, 12A, 12B, 13A, and 13B. FIG. 11A illustrates a bed B and a housing support frame 220. FIG. 11B is an enlarged view of the bed B and the housing support frame 220.

As illustrated in FIG. 11A, the bed-side support portion 200 further includes a housing support frame 220. The housing support frame 220 is sandwiched between the bed frame BF and the mattress M to be supported by the bed B.

In detail, as illustrated in FIG. 11B, the housing support frame 220 includes a base portion 221 and a housing support portion 222. The base portion 221 extends parallel to the top surface of the bed frame BF. The base portion 221 is sandwiched between the bed frame BF and the mattress M. The housing support portion 222 is combined with the tip end of the base portion 221.

The housing support portion 222 is disposed beside the bed frame BF. The housing support portion 222 extends downward from the tip end of the base portion 221. The lower part of the housing support portion 222 is bent in a U shape, and the housing support portion 222 has a tip part 222a extending upward.

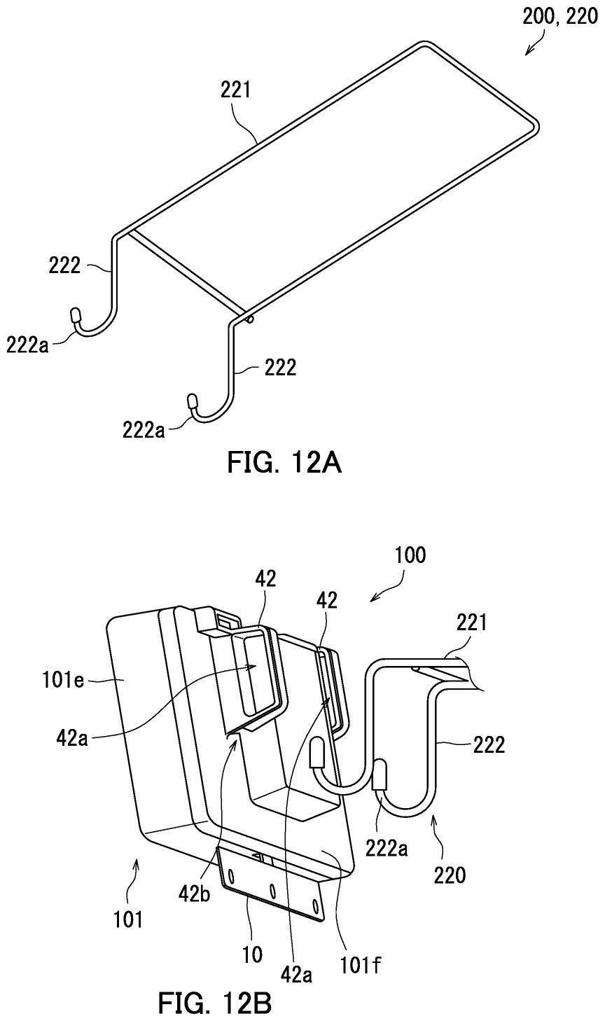

FIG. 12A is a perspective view of the housing support frame 220. As illustrated in FIG. 12A, in the present embodiment, the housing support frame 220 is made of a rod-shaped member. The housing support frame 220 has two housing support portions 222.

FIG. 12B is a perspective view of the urine amount measuring device 100 as viewed from the rear side thereof. More specifically, FIG. 12B illustrates a situation in which the housing support frame 220 is being attached to the second mounting portions 42 of the housing 101.

As illustrated in FIG. 12B, the housing support frame 220 is to be attached to the second mounting portions 42. In detail, the tip parts 222a of the two housing support portions 222a are inserted into the insertion holes 42b of the respective second mounting portions 42 described with reference to FIG. 2B. As a result, the housing 101 is hooked on the housing support frame 220 to be supported by the housing support frame 220.

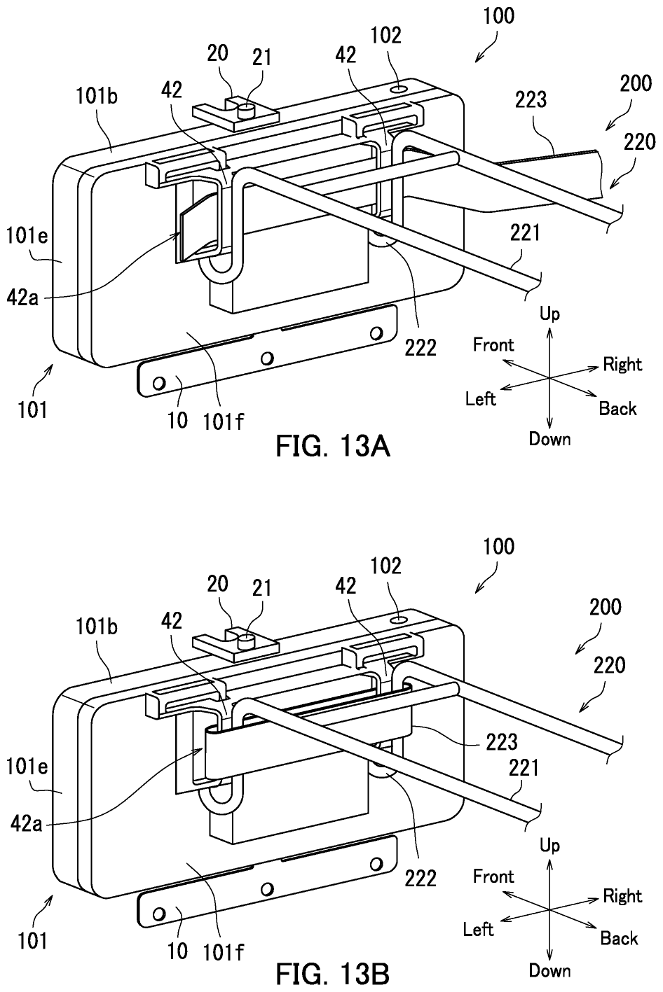

FIGS. 13A and 13B are perspective views of the urine amount measuring device 100 as viewed from the rear side thereof. More specifically, FIG. 13A illustrates a state in which a second belt member 223 is being attached to the two second mounting portions 42 of the housing 101. FIG. 13B illustrates the belt member 223 having been attached to the two second mounting portions 42 of the housing 101.

As illustrated in FIG. 13A, the bed-side support portion 200 further includes the second belt member 223. The second belt member 223 is attached to the second mounting portions 42. In detail, the second belt member 223 is inserted into the two second insertion holes 42a described with reference to FIGS. 2A and 2B. The second belt member 223 is a hook-and-loop fastener, binding tape, or a piece of string, for example.

As illustrated in FIG. 13B, the second belt member 223 fixes the two housing support portions 222 to the respective second mounting portion 42. As a result, the housing support frame 220 is fixed to the housing 101. Therefore, as described with reference to FIGS. 11A and 11B, the bed B can support the urine amount measuring device 100 by supporting the housing support frame 220.

As described above with reference to FIGS. 11A, 11B, 12A, 12B, 13A, and 13B, according to the present embodiment, the urine amount measuring device 100 can be supported on the bed B by using the housing support frame 220 even when the bed B does not have a fence FE.

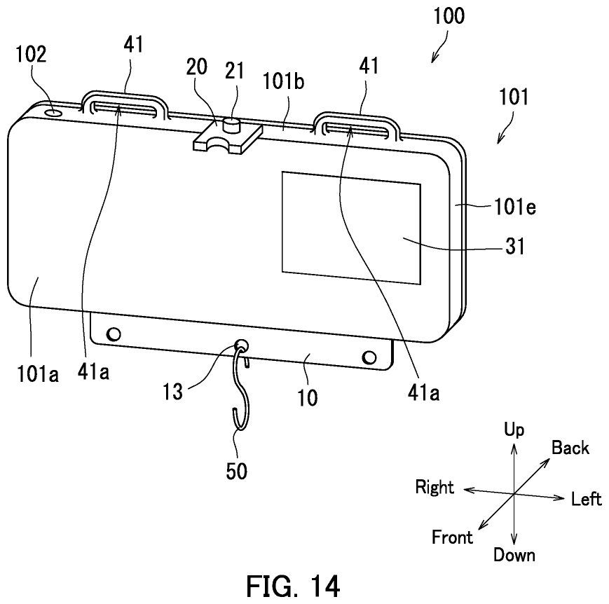

The following describes a variation of the urine amount measuring device 100 of the present embodiment with reference to FIG. 14. FIG. 14 illustrates a variation of the urine amount measuring device 100 of the present embodiment. In the urine amount measuring device 100 illustrated in FIG. 14, the two first mounting portions 41 are provided on the top surface 101b of the housing 101. The two first mounting portions 41 each protrude upward from the top surface 101b. As illustrated in FIG. 14, the first mounting portions 41 may be provided on the top surface 101b of the housing 101.

The urine amount measuring device 100 of the present embodiment includes the first mounting portions 41 and the second mounting portions 42, but the urine amount measuring device 100 may include only the first mounting portions 41 or the second mounting portions 42.

The first embodiment of the present invention has been described above with reference to the drawings (FIGS. 1 to 14). However, the present invention is not limited to the above embodiment, and can be practiced in various embodiments without departing from the gist thereof. In addition, the components disclosed in the above embodiment can be appropriately altered. For example, one component of all the components illustrated in one embodiment may be added to components of another embodiment, or some of the components of all the components illustrated in one embodiment may be removed from the embodiment.

The drawings schematically illustrate main components in order to facilitate the understanding of the invention. Aspects such as thickness, length, number, and interval of the components illustrated in the drawings may differ in practice for convenience of drawing preparation. Moreover, the configurations of each component illustrated in the above embodiment are merely examples and are not intended as specific limitations. The configuration of each component can be altered in various ways to the extent without substantial deviation from the effects of the present invention.

For example, in the embodiment described with reference to FIGS. 1 to 14, the first fixing portion 20 is connected to the center of the housing 101 in the left-right direction or the left end portion of the top surface 101b of the housing 101. However, the position where the first fixing portion 20 is connected to the housing 101 is not particularly limited. That is, the position of the connecting portion P is not particularly limited. The position of the connecting portion P may be any position according to the type of the urine storage container C.

The first fixing portion 20 is freely attachable to and detachable from the housing 101 in the embodiment described with reference to FIGS. 1 to 14. However, the first fixing portion 20 may not have to be freely attachable to and detachable from the housing 101. In other words, the first fixing portion 20 may be fixed to the housing 101. For example, the first fixing portion 20 and the housing 101 may be united. When the first fixing portion 20 is not freely attachable to and detachable from the housing 101, the urine amount measuring device 100 may include a plurality of the first fixing portions 20.

The housing 101 has two connecting portions P in the embodiment described with reference to FIGS. 1 to 14, but the number of the connecting portions P may be one, or three or more.

The first fixing portion 20 is connected to the top surface 101b of the housing 101 in the embodiment described with reference to FIGS. 1 to 14, but the position where the first fixing portion 20 is connected is not limited to the top surface 101b of the housing 101. For example, the first fixing portion 20 may be connected to the right side surface 101d or the left side surface 101e of the housing 101.

The container-side support portion 10 has three hooking holes 13 in the embodiment described with reference to FIGS. 1 to 14, but the number of the hooking holes 13 is not limited to three. The number of the hooking holes 13 may be one, two, or four or more. When the container-side support portion 10 has a plurality of the hooking holes 13, one of the hooking holes 13 is arranged at the center of the holding portion 12 in the left-right direction, and the others are arranged at locations separate in the left and right directions by an approximately equal distances from the center of the holding portion 12 in the left-right direction. Note that the hooking hole 13 located at the center of the holding portion 12 in the left-right direction may be omitted.

The housing 101 includes two first mounting portions 41 in the embodiment described with reference to FIGS. 1 to 14, but the number of the first mounting portions 41 is not limited to two. The number of the first mounting portions 41 may be three or more. When the housing 101 includes three or more first mounting portions 41, one of the first mounting portions 41 is arranged at the center of the housing 101 in the left-right direction, the others are arranged at locations separate in the left and right directions from the center of the housing 101 in the left-right direction by approximately equal distances. Note that the first mounting portion 41 disposed at the center of the housing 101 in the left-right direction may be omitted.

The holding portion H of the urine storage container C is hooked on one suspension portion 50 in the embodiment described with reference to FIGS. 1 to 14, but the holding portion H of the urine storage container C may also be hooked on a plurality of the suspension portions 50. That is, the urine amount measuring device 100 may include a plurality of suspension portions 50. When the urine amount measuring device 100 includes an odd number of suspension portions 50, one of the suspension portions 50 is hooked on the hooking hole 13 located at the center of the holding portion 12 in the left-right direction. The remaining suspension portions 50 are arranged at locations separated from the center of the holding portion 12 in the left and right directions by approximately equal distances. When the urine amount measuring device 100 includes an even number of suspension portions 50, the even number of suspension portions 50 are arranged at locations separated from the center of the holding portion 12 in the left-right direction by approximately equal distances in the left and right directions.

The suspension portion 50 is an S-shaped hook in the embodiment described with reference to FIGS. 1 to 14, but the suspension portion 50 is not limited to the S-shaped hook. The suspension portion 50 may have any shape as long as it capable of suspending the urine storage container C. For example, the suspension portion 50 may include a clip for griping the urine storage container C.

The sensor 32 is a load cell in the embodiment described with reference to FIGS. 1 to 14, but the sensor 32 is not limited to the load cell. The sensor 32 may be any device as long as it capable of outputting a signal that changes according to change in the force acting on the container-side support portion 10. The sensor 32 may be a pressure sensor, for example.

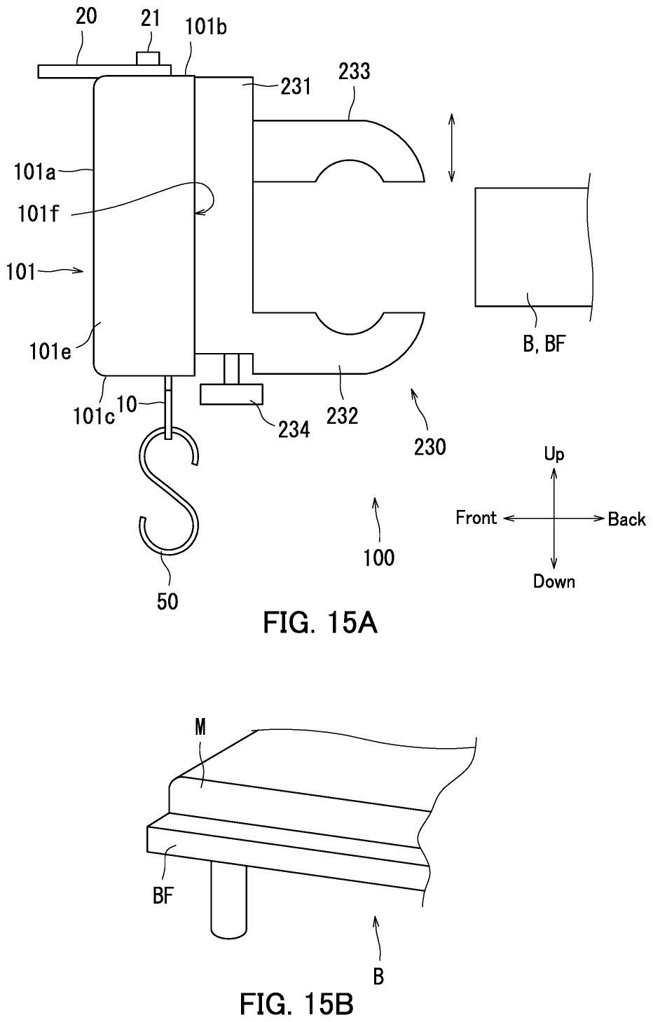

In the embodiment described with reference to FIGS. 1 to 14, mounting portions (the first mounting portion 41 and the second mounting portion 42) are provided on the housing 101 for mounting the housing 101 to the bed B. However, the configuration for mounting the housing 101 to the bed B is not limited to the above configuration. The following describes a second embodiment of the present invention with reference to FIGS. 15A and 15B. Note that the following describes the matters different from those of the first embodiment described with reference to FIGS. 1 to 14, and the same matters as those of the first embodiment described with reference to FIGS. 1 to 14 will be omitted. The configuration for mounting the housing 101 to the bed B in the second embodiment is different from that in the first embodiment described with reference to FIGS. 1 to 14.

FIG. 15A is a side view of a urine amount measuring device 100 of the second embodiment. FIG. 15B illustrates a part of the bed B. The urine amount measuring device 100 illustrated in FIG. 15A includes a gripping member 230 in place of the first mounting portions 41 and the second mounting portions 42 described in the first embodiment.

The gripping member 230 is provided on the rear surface 101f of the housing 101. The gripping member 230 grips the bed B. For example, the gripping member 230 grips the bed frame BF illustrated in FIG. 15B. Specifically, the gripping member 230 includes a base portion 231, a fixed piece 232, a movable piece 233, and an operating member 234.

The base portion 231 is fixed to the rear surface 101f of the housing 101. The base portion 231 extends in the vertical direction. The fixed piece 232 protrudes rearward from the lower end of the base 231. The movable piece 233 is provided above the fixed piece 232. The movable piece 233 extends rearward from the base 231. In other words, the movable piece 233 extends parallel to the fixed piece 232. The movable piece 233 is movable in the vertical direction.

An operator operates the operation member 234 to move the movable piece 233 in the vertical direction. Therefore, the operator operates the operation member 234 to keep the movable piece 233 away from the fixed piece 232 to a position where a first end of the bed frame BF can be inserted between the fixed piece 232 and the movable piece 233, and insert the first end of the bed frame BF between the piece 232 and the movable piece 233. Thereafter, the operator operates the operating member 234 to move the movable piece 233 close to the fixed piece 232. This can achieve firm gripping of the first end of the bed frame BF by the fixed piece 232 and the movable piece 233. As a result, the urine amount measuring device 100 is supported by the bed B.

### INDUSTRIAL APPLICABILITY

The present invention is useful, for example, in the medical fields. The present invention is also useful, for example, in the long-term care fields.

### REFERENCE SIGNS LIST

10: Container-side support portion
20: First fixing portion
32: Sensor
41: First mounting portion
42: Second mounting portion
50: Suspension portion
60: Second fixing portion
100: Urine amount measuring device
101: Housing
101b: Top surface
101f: Rear surface
200: Bed-side support portion
201: First belt member
210: Belt support member
220: Housing support frame
221: Base portion
222: Housing support portion
230: Gripping member
B: Bed
C: Urine storage container
P: Connecting portion
P1: First connecting portion
P2: Second connecting portion
T: Urinary catheter tube

1. A urine amount measuring device comprising:
a container-side support portion configured to support a suspension portion on which a urine storage container is to be hung, the urine storage container storing urine of a person lying on a bed;
a sensor configured to output a signal that changes according to change in force acting on the container-side support portion;
a housing that houses the sensor;
a first fixing portion configured to fix, to the housing, a tubular member that allows the urine to flow from the person to the urine storage container; and
a second fixing portion configured to fix the tubular member to the suspension portion.

2. The urine amount measuring device according to claim 1, wherein,
the container-side support portion supports the suspension portion in a freely swinging manner.

3. The urine amount measuring device according to claim 1, further comprising the suspension portion.

4. The urine amount measuring device according to claim 3, wherein,
the suspension portion supports the urine storage container in a freely swinging manner.

5. The urine amount measuring device according to claim 1, wherein,
the first fixing portion is detachably connected to the housing.

6. The urine amount measuring device according to claim 5, wherein
the housing includes connecting portions at a plurality of positions where the first fixing portion to be connected.

7. The urine amount measuring device according to claim 1, wherein,
the housing includes a mounting portion to be mounted on a bed-side support portion which is to be supported by the bed.

8. The urine amount measuring device according to claim 7, further comprising the bed-side support portion.

9. The urine amount measuring device according to claim 8, wherein,
the bed-side support portion includes:
a belt member configured to be attached to the mounting portion; and
a belt support member that is configured to be supported by the bed and from which the belt member is hung.

10. The urine amount measuring device according to claim 8, wherein,
the bed-side support portion includes a belt member to be supported by the bed,
the belt member is configured to be attached to the mounting portion.

11. The urine amount measuring device according to claim 9, wherein,
the mounting portion is provided on a rear surface of the housing.

12. The urine amount measuring device according to claim 9, wherein,
the mounting portion is provided on a top surface of the housing.

13. The urine amount measuring device according to claim 8, wherein,
the bed-side support portion includes:
a housing support portion configured to support the housing; and
a base portion configured to connect to the housing support portion and to be supported by the bed, and
the housing support portion is configured to be attached to the mounting portion.

14. The urine amount measuring device according to claim 1, further comprising a gripping member configured to grip the bed.

15. The urine amount measuring device according to claim 10, wherein,
the mounting portion is provided on a rear surface of the housing.

16. The urine amount measuring device according to claim 10, wherein,
the mounting portion is provided on a top surface of the housing.
